(19) United States
(12) Patent Application Publication
(10) Pub. No.: US 2026/0283558 A1
(43) Pub. Date: Sep. 24, 2026

(54) System for acquiring images in intraoperative magnetic resonance

(71) Applicant: Esaote S.p.A., Genova (IT)

(72) Inventors: Fabrizio Ferrando, Genova (IT); Paolo Maria Traverso, Genova (IT); Nicoletta Scati, Genova (IT); Lisa Ciubini, Genova (IT)

(21) Appl. No.: 19/473,612

(22) PCT Filed: Apr. 15, 2024
(86) PCT No.: PCT/IB2024/053680

§ 371 Date: Oct. 8, 2025

(30) Foreign Application Priority Data
Apr. 14, 2023 (IT) 102023000007260

(51) Int. Cl. A61B 5/00; G01R 33/38

(52) U.S. Cl. CPC A61B 5/704; A61B 5/702; G01R 33/3806

(57) ABSTRACT

AMENDMENTS TO THE ABSTRACT A system for acquiring images by intraoperative magnetic resonance includes a magnetic structure generating a static magnetic field and surrounding on at least partially a housing space for a patient's area, and a bed movable relative to the magnetic structure between an end position, where the bed is at least partially inside the housing space, and an outer end position, where the bed is outside the housing space and spaced from the magnetic structure. The bed is cantilevered by a column and is rotatable angularly, so that the bed can be continuously oscillated between an imaging position, where an internal end of the couch is positioned inside the patient's housing space with the patient's area of interest in the correct image acquisition position, and an outer position, where the same end of the couch is spaced from the magnetic structure and at a surgical operating position.

The present invention relates to system a for acquiring images by means of intraoperative magnetic resonance, in particular for the acquisition of progress state control images during brain surgery, the said system comprises a magnetic structure generating a static magnetic field and delimiting or surrounding on at least some sides a housing space for at least the patient's head and a bed that is movable in different relative positions with respect to the magnetic structure, and said positions being comprised between at least an end position in which said bed is at least partially inside the housing space and an outer end position in which said bed is outside said housing space and at a predetermined distance from the magnetic structure.

In the field of surgery, especially in the case of brain surgeries, it is provided to alternate phases of surgical execution with phases of verifying the conditions of the area where the surgical steps have been performed through the acquisition of images in nuclear magnetic resonance. In such cases, after steps of the surgical procedure or whenever the surgeon deems it appropriate, the surgical phase of the procedure is suspended and the patient is placed inside the housing space of an MRI apparatus for the acquisition of verification diagnostic images.

During brain surgery, the patient's head is firmly blocked to the bed in order to avoid displacement of the patient's head in relation to position references to which are also referred the positions of the operating instruments that, as known from the state of the art, require very precise positionings. Therefore, it is recommended that the patient is not transferred from an operating table that is dedicated to surgical activity and correspondingly equipped, to an MRI apparatus bed and vice versa whenever the surgical phase is interrupted, and control imaging is performed.

One solution therefore is that the MRI apparatus couch is configured to provide both functionalities, i.e. both that of an operating table and that of a patient positioning bed in the MRI apparatus. Typically, in this situation, it is provided that the bed may be moved alternately outside the magnetic structure and at a certain distance from it for the execution of the operation steps and inside the patient housing space for the acquisition of the control images. Since all vital-parameter monitoring units, drug-administration units and the systems and tools for carrying out the surgical steps, such as positioning and tool-guidance systems, have to be associated with the bed, in order to avoid interference with the magnetic field of the magnetic structure of the MRI apparatus-especially when this magnetic structure is of the type comprising permanent magnets-it is necessary to position the bed beyond a predetermined distance from the magnetic structure of the MRI apparatus, where the magnetic field generated by said permanent magnets has a very low intensity that does not affect the equipment and the tools required to perform the operation. The bed of the MRI apparatus must also be provided in combination with a so-called craniostat, i.e. a device for blocking the position of the skull which must remain firmly and fixedly coupled to the patient's skull for the entire duration of the operation.

A further aspect concerns the fact that the acquisition of MRI images requires a receiving coil for the magnetic resonance signals, which is preferably located close to the anatomical site to be examined and in this case to the head, so that the said coil must be alternately applied to the patient in the correct position for image acquisition and subsequently removed to allow access to the operating field.

A third important aspect for the execution of the aforementioned surgical modality comprising a sequence of alternating phases of surgery and control imaging is the duration of the surgery, which must be kept within predetermined time durations, so there is a need to limit the time required for image acquisition. In addition, it is also necessary to consider the time durations of the interruption periods between the individual surgical steps, which have to be as short as possible in order not to lengthen the overall surgical time exaggeratedly and not to excessively space out over time the individual surgical steps so as to reduce the overall duration of anesthesia.

In the case of image acquisition with nuclear magnetic resonance, the duration of the acquisition, especially in the case of soft tissue, is difficult to shorten, especially in order to acquire quality images that allow the condition of the surgical site to be detected. Therefore, in order to limit the duration of the intervals between one operation step and the next step in which the acquisition of the control image is performed, it is not possible or hardly possible to reduce the time of the acquisition process. In order to reduce the time interval between one operation step and the next, it is therefore necessary to reduce the transition time between the operation position for surgical activity and the patient's imaging position, which must allow for the rapid transfer of the patient and thus of the bed from an operation station that is external to the MRI apparatus and arranged beyond a predetermined minimum distance in order to avoid interferences of the magnetic field permeating outside the magnetic structure to the inside of the housing space of the MRI apparatus and vice versa.

In the state of the art there are several solutions describing the movement of magnets with respect to an operating station in order to perform brain surgery in which surgery steps are alternated with imaging steps of the operating field in order to monitor or verify the state of the field after the steps performed and to at least plan the next surgery step.

Devices for fixing the patient's head relative to the operating table during brain surgery are also known in the state of the art.

Dedicated receiving coils for nuclear magnetic resonance imaging of the head are also known, many of which are not compatible with the fixing devices for the position of the patient's head and therefore have to be applied to the patient's head for MRI image acquisition and removed during the operation steps, further increasing the time required.

Document US2010/102814 discloses a combination of and MRI apparatus and a patient table according to the preamble of claim 1.

In this document, there is discloses an MRI apparatus having an open U-or C-shaped magnet structure, wherein the magnet structure has at least one vertical connection member for joining two horizontal wall members which lie one above the other and which are supported in a cantilever fashion and in a predetermined spaced relationship by the said vertical member. The said the vertical member is eccentrically connected to the two wall members at a side edge thereof. The horizontal wall members and the vertical member delimit the upper and lower sides and at least a vertical lateral band of a space for receiving at least one part of a patient body. The horizontal wall members also support means for generating a static magnetic field that permeates the patient receiving space. The apparatus further includes a patient table, supported in an intermediate position between the two horizontal wall members, and lies slightly above the lower horizontal wall part, the table being displaceable in at least one displacement direction, having at least one component of motion towards and/or away from the vertical connection member, and the table being rotatable about a vertical axis outside the magnet structure, i.e. outside the horizontal wall members.

Document JP2000232969 discloses a combination of an MRI apparatus and a patient support table in which the patient support table is rotated around a vertical axis of rotation which is placed at an end of the table positioned outside the imaging gantry of the MRI apparatus. The rotation disclosed is around an angle which is of 90° as disclosed in FIG. 16.

Document DE19736884 discloses a combination of an MRI apparatus, a patient table which can be positioned alternatively inside the gantry of the MRI apparatus and outside of it by a rotational movement of the table in the horizontal plane. The end position of the patient table outside the gantry of the MRI apparatus is indicated of being at a certain distance from the magnetic structure of the MRI apparatus and the said combination of MRI apparatus and patient table is constructed in such a way that in order to have an end of the table at a certain distance from the magnets at which distance the magnetic flux diffusing outside the gantry of the MRI apparatus is lower than a certain level of intensity, a rotation of more than 90°, and specifically of 180° of the table is requested.

Document US2019/374417 discloses a medical system comprising a robotic operation table top, and a controller. The robotic operation table a robot arm supporting the table top. The controller gives the robot arm a command to move the table top to positions corresponding to the medical processes. The robot arm includes a plurality of joints, a plurality of movable elements connected by the plurality of joints to one another, and a plurality of electric actuators and a plurality of position detectors. The joints comprise two joints which are configured to connect two of the said elements in a horizontally-rotating way. The said two joints have each a different rotating axis which are positioned at a certain distance one from the other and particularly one at an end of the said table top and the other at a medial zone of the said table top. A patient lying on the said table top is positioned inside the gantry of an MRI apparatus and outside of it in a surgical operational station by a combined rotation of the said movable elements around both of the said axis. The said table top is also articulated to the said robot arm by means of a joint which allows to provide the said table top with different inclinations relatively to the horizontal plane.

Document U.S. Pat. No. 5,085,219 discloses an MRI RF coil holder for imaging temporomandibular joint. Elements are provided for positioning and securing the RF surface coil at a first predetermined point along a longitudinal axis of a patient positioning table and of the holder and for positioning and securing the RF surface coil at a second predetermined point transverse to the said longitudinal axis and at a radial distance R from the said longitudinal axis. The disclosed embodiment provides orbital movement of the surface coil about the said longitudinal axis using a pair of U-shaped runner guides disposed substantially transverse to the longitudinal axis and adjustable clamps in cooperative engagement with the runner guides. The surface coil can be adjusted along a path transverse to the said longitudinal axis by way of an adjustable shaft or arm which can be moved inwardly and outwardly, and the surface coil can be pivoted step-wise using a pivot head having a plurality of equally spaced detents. The pivot head is biased in a direction away from the surface coil and adjustably engages one of the plural detents while rotating about an axis substantially parallel to the longitudinal axis.

Document U.S. Pat. No. 5,735,278 discloses a patient support table having two opposite ends one of which is configured for forming a patient head positioning seat and the said end being formed by a terminal sector of the patient positioning table which is hinged to the rest of the patient positioning table in an oscillating manner around an horizontal axis.

The disclosed state of the art is such that the disclosed combination of features are designed in a way not to be able to reduce the time needed to transfer the patient from a surgical site into the gantry of an MRI apparatus and vice-versa, neither to have a simple and efficient construction reducing costs and not subjecting the service persons to complex burden in relation to manipulating the patient positioning devices.

The object of the present invention, therefore, is to realize a system for the acquisition of intraoperative magnetic resonance images, in particular in the context of head surgery and especially brain surgery, which system allows for faster and at the same time easier and more convenient MRI image acquisition operations during interruption intervals between two surgery steps of a succession of surgery steps.

In a first embodiment of the present invention, the said system comprises a magnetic structure generating a static magnetic field and delimiting or surrounding on at least some sides a housing space for at least the patient's head and a bed that is movable in different relative positions with respect to the magnetic structure, and said positions being comprised between at least an end position in which said bed is at least partially inside the housing space and an outer end position in which said bed is outside said housing space and at a predetermined distance from the magnetic structure and wherein
the said magnetic structure having a cross-section of a lying U-shaped or C-shaped type, wherein the said magnetic structure has at least one vertical connection element of two horizontal wall elements which are mutually vertically superimposed and are supported cantilevered and at a predetermined distance from each other by said vertical element, while said vertical element eccentrically connects at a lateral edge to said two wall elements, which said horizontal wall elements and said vertical element delimit at the top, at the bottom and over at least one vertical lateral section a housing space for at least one portion of a patient's body, and which horizontal wall elements carry static magnetic field generating means that permeates the said housing space,
wherein said patient support bed has one end external to the magnetic structure and provided at a predetermined distance therefrom and is supported cantilevered towards the opposite end by means of a column which is connected to the bed in the area of said external end and is rotatable about a vertical axis that coincides with or is parallel to the axis of said column thus enabling the bed to be continuously oscillated for a predetermined angular path, between two extreme positions, wherein the bed can be continuously oscillated between the said two extreme positions, in one of which, defined as the imaging position, the end of the bed opposite to the external end being provided with a patient's head positioning area and is positioned inside the patient's housing space of the said magnetic structure with the patient's head positioning area in the correct image acquisition position relatively to the magnetic structure, and in the other of those extreme positions, the said end opposite to the said external end of the said patient support bed is positioned at a certain predetermined distance from the magnetic structure and at the position of a surgical operating station and wherein
the said angular path of oscillation of the said patient support bed is less than 90° and around only the said vertical axis of rotation.

It is possible to provide different constructive variants of this configuration to achieve similar functionality.

Construction variants can provide alternatively or in combination and freely selectable a manual movement of the bed along the angular path and/or motorized drive elements can be provided in combination with activation and stop commands of said drive elements.

According to an advantageous embodiment, the distance of the rotation axis with reference to the central vertical axis of the magnetic structure is selected to such a measure, so that when the bed is rotated in the position inside the magnetic structure, the said patient's head positioning area is positioned relatively to the said magnetic structure such as to be coincident with a so-called imaging volume, i.e. a portion of the overall volume of the patient's housing space in which the static magnetic field generated by permanent magnets has sufficient homogeneity characteristics in order to obtain useful, high-quality diagnostic images and thus positioning the head of a patient lying on the said patient support bed in a position coincident with the said imaging volume.

Furthermore, in combination with the above, said distance and angular amplitude of rotation of the bed about the vertical rotation axis are selected in such a measure that, in the said extreme external position, the bed end associated with the patient's head positioning area can assume a position with respect to the magnetic structure such that, in this position, the influence of the static magnetic field permeating off said magnetic structure lies below a predetermined intensity value and therefore does not generate interference with the equipment and operating units whose presence is required for the execution of the operation.

According to an embodiment, the said extreme externa position of the patient support bed at which the end of the said patient support bed at which the patients head positioning area is provided is coinciding with an operating position in a surgical operation station and at which extreme position of the said patient's head positioning area and of the said surgical operation station the intensity of the magnetic field permeating out of the gantry of the the MRI apparatus or of the magnetic structure of the said MRI apparatus is out of the 5 Gauss equipotential line of the magnetic field generated by the said magnetic structure, this meaning that the said intensity of the magnetic field is preferably not higher than 5 Gauss or lower than 5 Gauss as specified in FIG. 3 of the annexed drawings.

In order to stabilize the bed cantilevered from the column, the column can be equipped with ballasts to counterbalance the weight of the bed cantilevered over the column and/or provide a foot for the column and secure it to the floor.

Alternatively or in combination it is also possible to insert a support equipped with wheels in an area of the cantilever bed to increase its stability and limit its deflection in the area inside the gantry. This support can be integral with the bed or possibly removable.

In addition to the option of performing angular movements, it is possible to provide for the bed to perform movements according to further degrees of freedom.

Alternatively or in combination with the above, according to an embodiment the bed is vertically liftable and lowerable, for example by providing that the support column is extendible and shortenable telescopically. The lengthening and/or shortening of the column can advantageously be driven by a dedicated gearmotor unit or a linear actuator such as an electromechanical linear actuator or a hydraulic or pneumatic actuator.

According to a further feature which can be provided in combination with one or more of the previous features, the bed may be realized extendible and shortenable and/or linearly translatable with respect to the support column, thereby varying, according to the user's choice, not only the overall length and/or the circumferential path of the end of the bed intended to enter the magnetic structure, but also the position of the end of the bed associated with the head with respect to the column.

Still according to a feature that can be provided in any combination or sub-combination with the above-mentioned features, the bed can also be supported in such a way that it can oscillate around a substantially horizontal axis, i.e. from top to bottom and vice versa.

For this purpose, between the bed and the end of the coupling column to the bed, an oscillation joint may be provided according to at least one axis, in particular but not exclusively according to at least one axis oriented transversely with respect to the longitudinal extension of the patient support bed. The oscillation may take place either by moving upwards the end opposite to the column or vice versa by moving said end downwards.

Still according to a further characteristic, which may be provided alternatively to each of those previously mentioned or in any combination or sub-combination with those previously mentioned, the end zone of the bed opposite to the column is formed by a terminal section which is hinged in an oscillating manner about a horizontal axis and orthogonal with respect to the longitudinal extension of the patient support bed, so that said terminal section oscillates about said horizontal axis upwards and/or downwards with respect to the remaining part of the bed.

All of the movements described above can be controlled by a motorized actuator and/or a hydraulic or pneumatic actuator and can be executed at least partially simultaneously or in sequence and/or slightly partially overlapping each other for each element. Due to the movements described above, the bed can not only be easily and quickly moved from the surgical station to the imaging station and vice versa, but it is also possible to quickly and easily carry out positionings according to multiple degrees of freedom, such as moving the bed from the level at which the surgeon is comfortable in the surgical station, and conversely moving the said bed level to a position vertically coinciding with the bed's housing space in the magnetic structure. Furthermore, the possibility of tilting the entire bed, or at least the part associated with the patient's head, in relation to the horizontal position makes it possible to regulate the blood flow to the brain by decreasing it, for example, in order to make it easier for the surgeon to view the surgical field.

According to an embodiment, a position fixing device of the patient's head to the bed, referred to as craniostat for brevity, is stably or replaceable attachable to the bed, said head position fixing or restraining device or craniostat being provided in combination with an MRI signals receiving coil, said receiving coil being formed by one element or by several elements which are electrically connected to each other, which element or elements are removably attachable to at least part of the structural elements of the craniostat at predetermined points thereof so as to configure in the condition attached to craniostat a receiving coil for the head.

In a variant embodiment, the craniostat consists of an anatomical support base of the nape of the patient's head and at least of two curved lateral elements that extend from said support base towards a corresponding temple of the patient and at a predetermined point of the temporal area have skull clamping organs that are movable in a clamping position and in a releasing position of the patient's head, while the base and/or side elements have hooking and/or fitting seats for one or more coil elements formed as solid plates and/or annular plates which incorporate the conductors forming the coil and the conductors of each plate end and begin in a connector terminal for jumpers that connect to each other the conductors of said plates so as to form the complete circuit of the coil from the individual circuits provided in the individual plates.

Alternatively, two wings or two coil half-shells are hinged to the base of the craniostat for the patient's head on its two temporal sides in order to oscillate between an open coil position in which said wings or half-shells are laterally swung out towards the bed and a closed position, wherein said wings or half-shells are oscillated against each other so as to form a cup-shaped element that surrounds the patient's skull, the two wings or half-shells respectively accommodate a segment of the coil conductors and have at their edges facing each other in the closed state terminals for a temporary and separable connection of mechanical and electrical type, through which the conductor segments of the two wings or the two half-shells are connected together in an electrically conductive manner to form the complete coil circuit, and also in such a way that the two wings and the two half-shells are mechanically connected to each other in a removable manner. The two half-shells do not necessarily have to be mechanically connected to form a single receiving channel but can form surface receiving channels that are independent and topologically complementary.

According to a variant, a receiving coil segment may consist of at least one circumferential band of the skull support base and comprise segments of coil conductors extending from one to the other of the hinge sides of said wings or half-shells, wherein at the ends of said coil segments are provided electrical terminals which are flexible or movable in the direction of oscillation and connect to corresponding terminals of the coil segments in said wings or in said half-shells and provided in a position coinciding with said flexible terminals at the ends of the coil conductor segments in said housing base.

The aforementioned embodiments are very variable, always remaining within the context of the concept of considering a coil circuit integrated into a structure that is either constrained in an openable manner to the craniostat or consists of one or more elements of a structure that can be attached to and removed from the craniostat structure.

According to a variant embodiment the receiving coil has an annular shape and is fixable to the head of the patient and/or the craniostat by means of clamping bands or belts, the inner diameter of the annular shape and the shape in plan being configured to contain within the opening at least the operation field, making it accessible even when the receiving coil is worn on the head of the patient.

Still according to an embodiment that can be provided in any combination or sub-combination with one or more of the above-mentioned embodiments and/or variants, since during the operation the patient is monitored by several physiological parameter monitoring units which detect these parameters by means of probes attached to the patient, and since during the operation the patient is administered one or more drugs of a different nature, the patient is connected to said monitoring units and/or units for dosing and dispensing said drugs by means of a plurality of electrical cables and/or fluid supply hoses, the bed is provided along at least part of its surface extension and/or along at least part of its perimeter edges with elements for removable fastening or retaining of said cables and/or tubes, and said cables and/or said tubes are provided with a redundant length that allows them to follow the movements of the bed and in particular the rotation between said two extreme positions.

In one preferred embodiment, the patient support bed may be provided with one or more housing channels having a closed annular cross section or an open cross section being provided with closure elements of the entire length of the longitudinal extension of the said channel or of part of the said length. The said housing channels being provided at least on one longitudinal side, in a position adjacent to at least part of its lateral edge or integrated in at least part of at least one of the lateral edges of the said bed or along the bottom side of the said bed and at one or both of the said lateral edges.

According to a further possible alternative, the patient support bed may be provided integrated inside its structure.

In each of the above disclosed alternatives the structure of the bed and/or the walls of the said one or more channels may comprise electric and/or electromagnetic shields avoiding that the said cables or tubing may interfere with the magnetic field and/or with the transmission and/or receiving coils of the MRI apparatus.

In this way and also thanks to the angular displacement of the bed it is possible to overcome the problems concerning the fact that the handling of the bed with the patient on it, in the condition required to perform the operation can lead to excessive lengths of the said cables and/or tubes in order not to limit the relative distance between the surgical station and the imaging station, so as to reduce below a predetermined threshold the intensity of the magnetic field permeating out of the magnetic structure, or to envisage excessive redundancy lengths of said cables and/or tubes, including the cable connecting the receiving coil to the machine.

Further features of the invention are the object of the dependent claims.

These and other features and advantages of the present invention will appear more clearly from the following description of some exemplar embodiments illustrated in the attached drawings, in which:

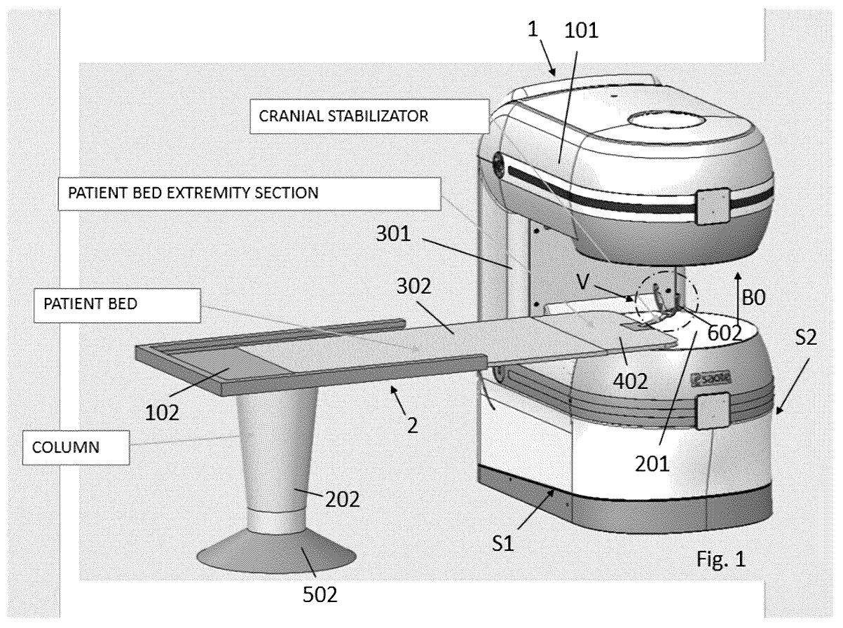

FIG. 1 shows a perspective view of an MRI system for performing intraoperative imaging according to the present invention, i.e., for performing MRI image acquisitions in workflow intervals of the surgery steps.

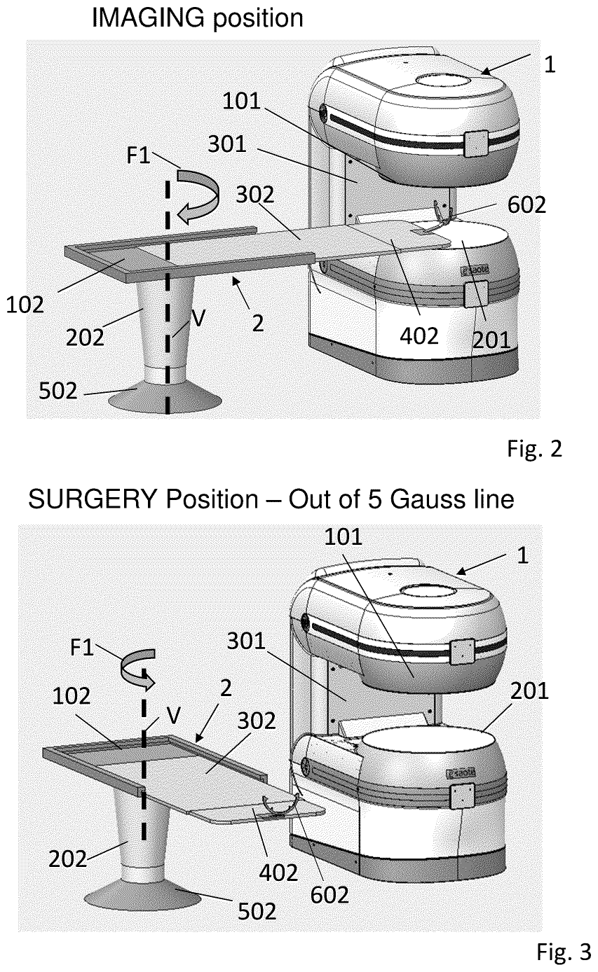

FIGS. 2 to 9 show different degrees of freedom for the movement of the bed relative to the magnetic structure of the MRI apparatus, which can be provided in combination with the rotation of the bed in FIGS. 2 and 3 alternately or in combination with each other.

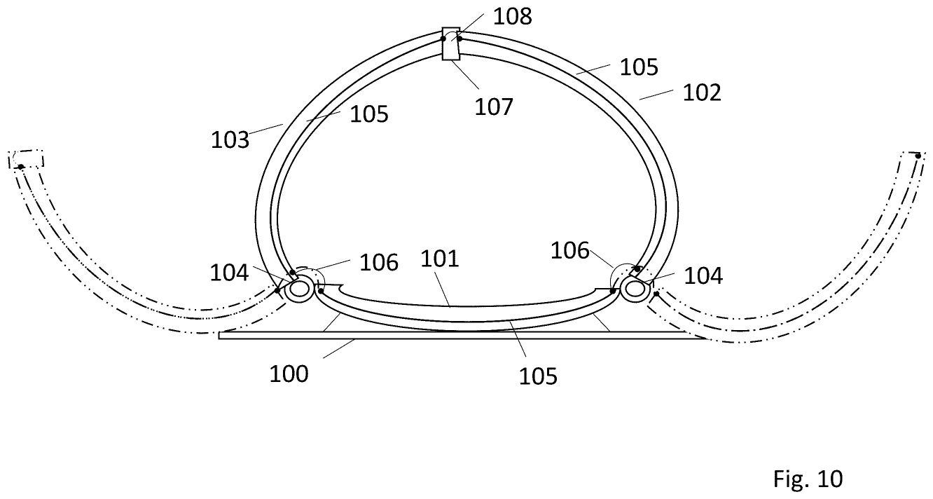

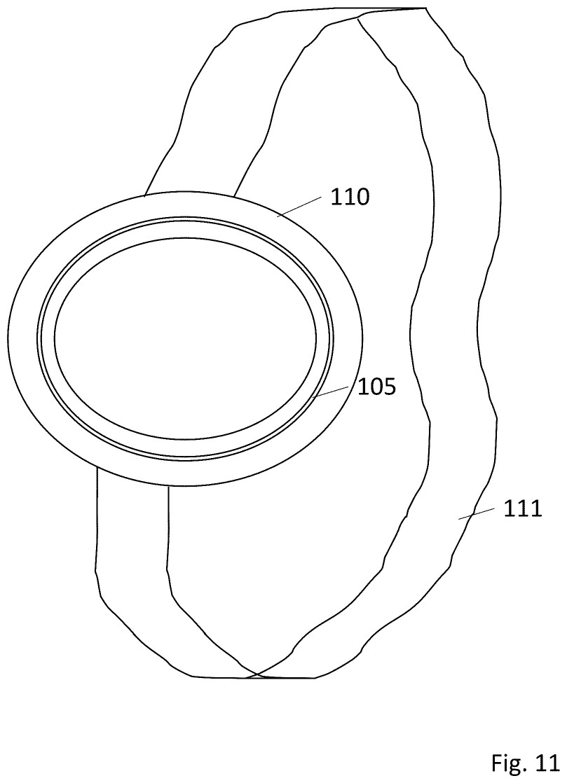

FIGS. 10 and 11 show two alternative embodiments of the receiving coil for the head.

With reference to the figures, a machine for image detection in nuclear magnetic resonance comprises a magnetic structure 1 and a patient support bed 2. In this description as well as in the introduction and claims, for the sake of simplicity, reference is made to the magnetic structure when speaking generally about the part consisting of the yoke, the means for generating the static field, the poles and the organs normally provided in such machines, such as gradient coils, compensation coils, electromagnetic shields, temperature measurement and/or temperature regulation means and other organs, and further consisting of the covering to protect these organs from the outside, which, as will be seen later, is made up of wall elements forming parts of an external covering shell with the functions of protecting the machine, patients and users and having additionally aesthetic and/or fixing functions for parts that do not contribute to the excitation and reception of signals, from which the image is generated. This simplification should be clear and unequivocal to for those skilled in the art, so that the definition “magnetic structure” as a “pars pro toto” term is to be considered equivalent, as it essentially determines the shape and dimensions, while the additional organs are generally carried by the said structure and only determine thickness increases and reductions of the housing cavity CV for the patient.

It is also worth noting that in the present invention reference is made to a magnetic structure in which the two poles are perfectly horizontal, as well as the patient-carrying plane of the bed, although it is also possible to provide the same structure in which the poles are inclined to some extent relative to the horizontal plane or in which said poles are not plane.

With reference to the figures and as stated above, the magnetic structure or magnet of an MRI machine has a C or inverted U shape with two horizontal branches that are kept spaced apart from each other on one side by a vertical element 301. The two horizontal branches form wall elements 101, 201 that delimit between them, both above and below, a housing space CV for a patient or a part thereof. In particular in the illustrated figures the distance between the two branches of the magnetic structure is such that it allows the introduction of even the trunk portion of the patient into the housing space or cavity CV for the patient. These two branches are formed by horizontal branches of a yoke that carry, on mutually facing sides, means for generating the magnetic field, such as layers of magnetized material, poles consisting of plates of ferromagnetic material, various coils for generating variable magnetic fields, the so-called gradient coils, the coil for transmitting excitation pulses of nuclear spins, and other operational units, such as shielding means, temperature measurement means and other means. Between the two horizontal branches, a static magnetic field is generated, indicated as Bo. The vertical wall element 301 is composed of a vertical yoke element and additional layers of magnetized or ferromagnetic material that contribute to optimizing the static magnetic field Bo. All these elements are covered by an external protective shell that gives the machine an aesthetically pleasing shape.

The static magnetic field Bo is not qualitatively ideal for the image acquisition throughout the entire extent of the horizontal wall elements 101 and 201, whereas it is possible for it to meet the desired features, especially uniformity, in a partial volume of the overall volume of said cavity that is delimited by an ideal spherical or ellipsoidal surface and is referred to as the imaging volume, being schematically illustrated and denoted as V in the figures.

The magnetic structure is provided in combination with a bed 2 that has a substantially horizontal patient-carrying plane 102, which is cantilevered at one end external to the magnetic structure 1 by a vertical column 202 which is appropriately equipped with ballasts or fixed to the floor, as indicated by the foot 502, to prevent the bed from tipping when loaded with a patient.

The column has a height or can be brought to a length such that the bed extends between the two horizontal walls 101 and 201, particularly at an upper level of the lower horizontal wall element 201. In this way, the bed can be inserted into the patient housing cavity CV with a transverse movement to its longitudinal extension, in such a way that the same is cantilevered into the patient housing cavity.

With reference to the illustrated configuration, one of the support elements of the patient-carrying plane 102 is designed in such a way as to allow rotation of said patient-carrying plane 102 around a vertical axis V and perpendicular to the bed support plane. The arrangement of this axis is such that the rotation axis coincides with the end of the bed coupled to the column 202, the latter being designed so that the coupling end consists of a seat accommodating a joint for fixing to the bed that rotates around said vertical axis V.

The column 202 is positioned externally to the magnetic structure and in particular in a position flanking one of the sides of the magnetic structure which are oriented parallel to a plane of vertical section intersecting the horizontal branches and the vertical branch of said magnetic structure and along which the branches of the C-or U-shaped section of said magnetic structure extend.

In the following description, these sides will be referred to briefly as the lateral flanks S1 and S2 of the magnetic structure, while the two other sides will be referred to as the open frontal front side and the closed rear side.

The axis of oscillation is positioned in such a position as to lie within the projection of the width dimension of the upper and lower horizontal wall elements 101 and 201 in the direction perpendicular to said lateral flanks, and at such a distance from the corresponding lateral flank of the magnetic structure that the vertical support element 302 of the patient-carrying plane 102, located at the opposite end of said patient-carrying plane, is laterally adjacent to the opposite lateral flank S1 of the magnetic structure and during the oscillation of the bed, it moves along a concentric circular path with the oscillation axis O, which is external to the magnetic structure, thus allowing the patient-carrying plane to enter the patient housing cavity CV substantially from the open front side of the magnetic structure, thus bringing also the patient into said cavity.

As will be clearer from the following description, in addition to the rotational movement of the bed relative to the magnetic structure, it is possible to foresee additional degrees of freedom for the bed, both alternative to each other and in any combination or sub-combination of said degrees of freedom.

A possible embodiment may involve the foot 502 being designed as a carriage with wheels that are lockable or retractable relative to the foot itself, allowing the activation and deactivation of the wheels and thus making the foot 502 and therefore the vertical supporting element 202 alternatively movable and lockable in position. Preferably, the said foot 502 may be equipped with a carriage constrained to a guide or rail fixed to the floor.

This allows the easy adjustment of the bed's position, particularly the vertical support element 202 of the bed, as well as the position of the vertical rotation axis of the same relative to the magnetic structure, being therefore possible to adapt the imaging system to different configurations of stations external to the system and intended to accommodate the operational units necessary for carrying out surgical activities.

In particular, the position of the rotation axis relative to the central vertical axis of the magnetic structure is chosen so that, with said rotation, the bed, and especially the end opposite to the one constrained to the column 202 and carrying the patient's head fixing device 602, can be brought with said rotation into the patient housing cavity of the magnetic structure 1 and in the position coinciding with the imaging volume V and in a position coinciding with the surgical station, and in particular at such a distance that the said station and the end of the bed 2 associated with the position stabilization and fixation device for the patient's head are sufficiently far from the magnetic field permeating outside the magnetic structure, especially when this is of the type whose magnetic field is generated by permanent magnets.

In particular the distance of the surgical station, not illustrated, should preferably be such that it is positioned beyond the magnetic field intensity line of 5 Gauss.

FIGS. 2 and 3 show the rotation of bed 2 relative to magnetic structure 1 and approximately indicate the two bed rotation positions, in which the bed and consequently the patient are positioned inside the housing cavity of magnetic structure 1 (FIG. 2) and outside of it in the surgical station, beyond the field line corresponding to the 5 gauss intensity of the magnetic field (FIG. 3). The rotation of bed 2 around axis V is indicated by the arrow F1.

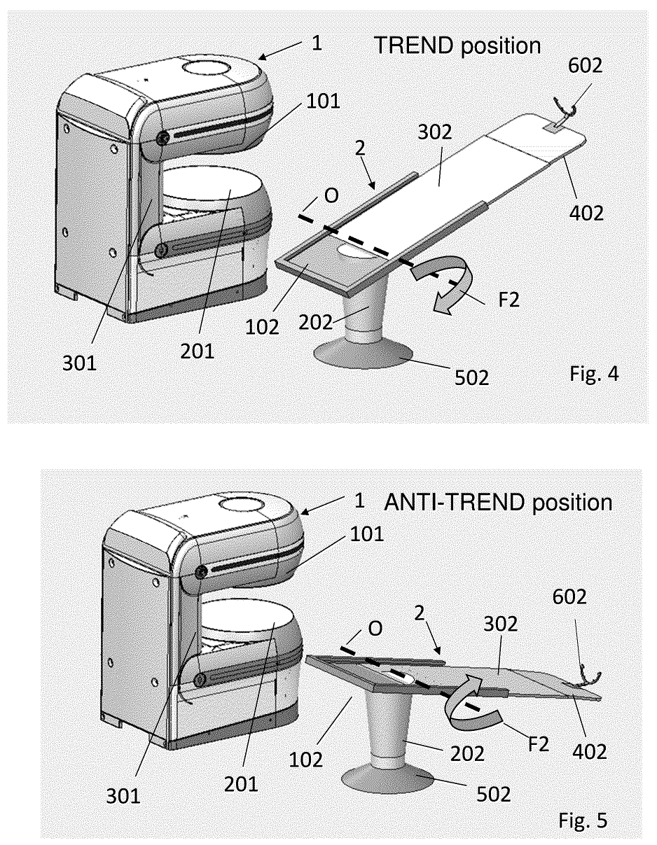

As illustrated in FIGS. 4 and 5, the bed can be supported by column 202 also thanks to coupling joints that allow at least one additional degree of freedom of movement. In FIGS. 4 and 5, the coupling to the column 202 of the bed takes place by also providing a swivel joint along a horizontal axis, as indicated by arrow F2 and axis O.

This can occur thanks to a universal joint, one of whose elements is fixed at the top of the rotation joint around the vertical axis V, while the other is fixed to bed 2, or through other types of coupling joints.

The bed 2 can thus be oscillated relative to the horizontal position both upward and downward, i.e., with the patient's head at a higher level than the feet or with the patient's head at a lower level than the feet. Such positions are advantageous, for example, when it is desired to reduce blood flow to the surgical site or when the best posture for the surgeon is at a higher or lower level than that planned to allow the bed to enter the housing cavity of the patient in the magnetic structure.

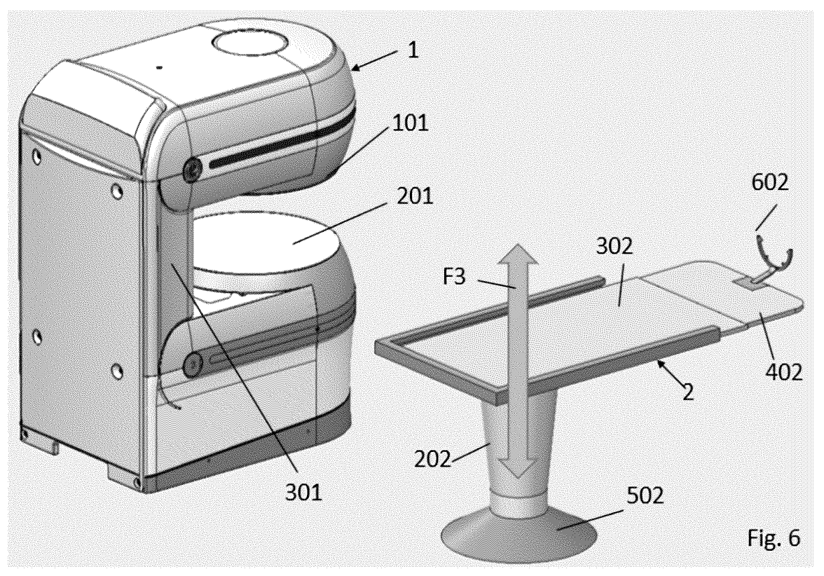

With reference to FIG. 6, it is possible to consider an additional third degree of freedom related to the height position of the bed from the floor while maintaining its horizontal orientation. Obviously, the lifting and lowering of bed 2 according to this third degree of freedom is also possible in combination with the second degree of freedom that allows the inclination of the bed relative to the horizontal orientation.

The lifting and lowering of bed 2, as indicated by arrow F3, can be achieved through a column 202 with a telescopic configuration that can be extended and shortened.

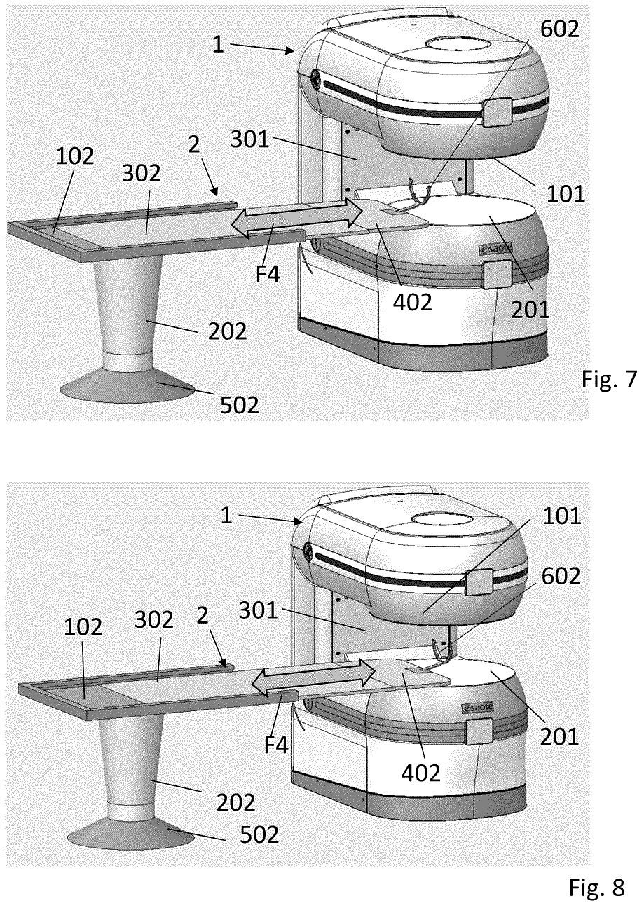

As indicated in FIGS. 7 and 8, alternatively or in combination with one or only some or all of the movements of bed 2 made possible according to the various degrees of freedom described above, it is possible to have an additional fourth degree of freedom that allows the translation of the bed 2 plane relative to the column 202 and/or the lengthening or shortening of the bed along its longitudinal axis.

The variation in the position of the bed relative to the column 202 along the longitudinal axis of the bed can take place thanks to a slide, for example, fixed at the upper end of the column 202 and slidingly engaged in a guide or track fixed to the lower side of the bed.

In this case, the length of the bed remains fixed.

In the illustrated case, however, the bed is realized in at least two parts that are mutually translatable in the direction of the bed's longitudinal axis, such as a telescopic configuration of bed 2 itself.

In this case, the overall length of bed 2 varies. This fourth degree of freedom is indicated by arrow F4.

As is evident, this degree of freedom allows the patient's head to be brought into a position perfectly centered and coincident with the imaging volume V, and also in a position precisely coincident with that envisaged for the patient's head at the surgical station.

The translation F4 of the bed, whether in the form that changes its position relative to the column 202 or in the variant that involves a variation in the length of the bed, allows placing the surgical station further away from the magnetic structure, optionally even closer, for example, to limit the action.

Since the relative position between the surgical station and magnetic structure 1 depends on the intensity Bo of the field generated by the magnets, or in case to increase the distance between the surgical station and magnetic structure to further reduce the influence of the magnetic field permeating outside of it, or to maintain a position beyond the 5 Gauss intensity line, adapting to possible higher intensities of the static magnetic field.

According to a possible embodiment variant provided alternatively or in combination with one or more of the variants related to the degrees of freedom from the second to the fourth as described above, the bed 2 plane that supports the patient is divided into two parts, one of them, 402, is located at the end of the bed opposite to the one that connects to the column and consists of a terminal band of the bed carrying the patient fixing device 602.

The mentioned terminal part 402 is pivotally hinged around a horizontal axis according to arrow F5, allowing the positioning of the patient's head in a raised position compared to the rest of the body.

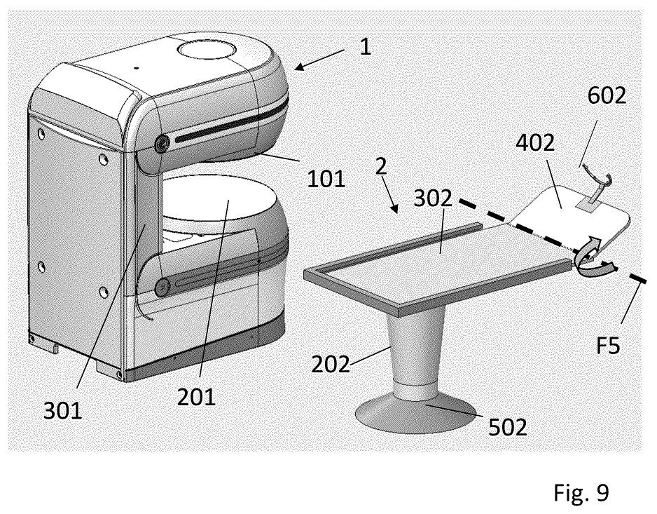

As shown in FIG. 9, the fixing device 602 for the head is cantilevered beyond the terminal edge of the corresponding end of the bed, for example, by means of a bracket. The length of the bracket is such that the said end is positioned at the level of the shoulders or at a level lower than that of the shoulders.

Regarding the movements according to the different degrees of freedom, these can be both manually operated and also preferably motor-driven. These can be of the electric and/or hydraulic and/or pneumatic type, being provided in combination with a control unit, preferably programmable and controllable to carry out the movements of the bed through a control interface.

To make the translation operations of the bed between the surgical station and imaging positions easier, it is possible to implement commands that already include a combination of movements according to two or more degrees of freedom, executed successively, simultaneously, or partially simultaneously. In this case, it is possible to automatically complete the positioning of the patient from one position to another, allowing the staff to manually adjust the patient's position as needed.

Of course, the control interface can be implemented both with a cable connection and a wireless connection, such as via Wi-Fi, Bluetooth, or similar, or it is also possible to consider a voice-controlled interface and/or gesture-controlled interface.

FIGS. 10 and 11 show by way of non-limiting examples two possible embodiments of a receiving coil particularly suitable for intraoperative imaging that allows for quick coupling in the surgical position to the patient's head.

FIG. 10 shows a cross-sectional view of a craniostat according to a vertical transverse plane of the bed, showing a fixing plate 100 carrying the anatomical base for supporting the patient's head, indicated as 101. At the lateral ends of the said anatomical base 101, two wings 102, 103 are hinged at 104, forming two segments of the receiving coil.

Conductors forming the coil circuit and indicated as 105 are embedded in both the anatomical base 101 and the wings 102, 103. The conductors embedded in the wings 102 and 103 are connected through flexible connecting conductors 106 to the conductor embedded in the support base 101, while the free ends of the wings 102, 103 can be fixed together in the closed and operational configuration of the coil by mechanical devices for a removable coupling indicated generically as 107. Additionally, the conductor segments 101 in the two wings can be connected, when required, also at the corresponding ends by means of electrical connection terminals indicated as 108.

The conductor segments of the wings 102, 103 are connected to the segment of the support base 101 at the hinges 104, for example, through flexible conductors or movable electrical couplings.

The open condition of the coil, allowing accessibility to the operation site, is shown with dashed lines, as well as the condition of the flexible connection terminals 106 of the conductor segments 105 at the hinges 104.

Alternatively to the above, the coil can be formed by multiple plates or modules that can be removably attached to each other and contain conductor segments forming the receiving coil circuit, where these elements can be removably coupled to each other and to the base plate and/or the patient's head fixing device through mechanical couplings that can be coupled and decoupled. In combination, terminals are provided for the electrical connection of the conductor segments of the coil circuit, which can also be coupled and decoupled from each other. Preferably, the mechanical coupling devices and the electrical coupling devices are integrated with each other, allowing simultaneous mechanical and electrical coupling of the elements forming the coil.

FIG. 11 shows a possible additional variant of a coil indicated as 110, consisting of an annular-shaped coil that can be fixed to the patient's head in various positions using a fastening strap 111, for example shaped and sized to be placed with the opening centered on the surgical site, with the annular coil surrounding it while maintaining accessibility for the surgeon.

Those described are only two non-limiting examples, but it is of course also possible to use non-opening and/or rigid coils in the system according to the invention. For instance, it is possible to provide a rigid coil equipped with a mechanical attachment system integrated on the top of the patient support.

As evident, the above contribute to make the patient easily and quickly movable between a surgical station and a diagnostic apparatus by magnetic resonance imaging, thereby contributing to reduce the overall surgery time and to make the MRI image acquisition operations safer and more comfortable.

The invention claimed is:

1. A system for acquiring images by intraoperative magnetic resonance imaging, including fir acquiring progress state control images during brain surgery, the system comprising:
a magnetic structure generating a static magnetic field and delimiting or surrounding, on at least some sides, a housing space for at least a patient's head; and
a bed that is movable in different relative positions with respect to the magnetic structure, said positions being comprised between at least an end position, in which said bed is at least partially inside the housing space, and an outer end position in which said bed is outside said housing space and at a predetermined distance from the magnetic structure,
wherein said magnetic structure has a cross-section that is U-shaped or C-shaped, in which the magnetic structure has at least one vertical connection element of two horizontal wall elements which are mutually vertically superimposed and are supported cantilevered and at a predetermined distance from each other by said vertical connection element, said vertical connection element eccentrically connecting, at a lateral edge, to said horizontal wall elements, said horizontal wall elements and said vertical connection element delimiting at a top, at a bottom and over at least one vertical lateral section a housing space for at least one portion of a patient's body, said horizontal wall elements carrying static magnetic field generating means that permeate said housing space, and
wherein said bed has one end external to the magnetic structure and provided at a predetermined distance therefrom, said one end being supported cantilevered towards an opposite end by a column which is connected to the bed in an area of said external end and is rotatable about a vertical axis that coincides with or is parallel to an axis of said column,
thereby enabling the bed to be continuously oscillated for a predetermined angular path between two extreme positions,
wherein in one of the two extreme positions, defined as an imaging position, an end of the bed opposite the external end being provided with a patient's head positioning area and said external end being positioned inside the patient's housing space of the magnetic structure with the patient's head in correct image acquisition position with respect to the magnetic structure, and in another one of the two extreme positions, an end opposite to the said external end positioned at a predetermined distance from the magnetic structure and at the position of a surgical operating station, and
wherein the angular path of oscillation of the bed is less than 90° and around only the vertical axis of rotation.

2. The system according to claim 1,
wherein a distance of a rotation axis with reference to the vertical axis of the magnetic structure is selected to an extent causing, when the bed is rotated to the position inside the magnetic structure, the patient's head positioning area is positioned relatively to the magnetic structure such as to be coincident with an imaging volume, the imaging volume comprising a portion of an overall volume of the housing space of the patient, in which the static magnetic field generated by permanent magnets has sufficient homogeneity characteristics in order to obtain high-quality diagnostic images and thus positioning the patient's head lying on the bed in a position coincident with the imaging volume, and
wherein said distance and angular amplitude of rotation of the bed about the vertical axis are selected to cause, in an extreme external position, a bed end associated with the patient's head positioning area can assume a position with respect to the magnetic structure such that an influence of the static magnetic field permeating off said magnetic structure lies below a predetermined intensity value and does not generate interference with equipment and operating units whose presence is required for execution of surgery.

3. The system according to claim 1, wherein the bed is supported to be vertically liftable and lowerable by bi-directional lifting means.

4. The system according to claim 1, wherein the bed is provided with a means configured for extending and shortening, or linearly translating the bed with respect to the column, thereby varying, according to a user's choice, an overall length and/or a path of the end of the bed intended to enter the magnetic structure, further varying a the position of the end of the bed associated with the head with respect to the column.

5. The system according to claim 1, wherein the bed is provided with rotatable support joints configured so that the bed can rotate about a substantially horizontal axis, from top to bottom and vice versa.

6. The system according to claim 1, wherein an end zone of the bed opposite to the column is formed by a terminal zone, which is hinged in an oscillating manner about a horizontal axis and orthogonal with respect to a longitudinal extension of the bed, whereby said terminal zone oscillates about said horizontal axis upwards and/or downwards with respect to a remaining part of the bed.

7. The system according to claim 1, wherein movements of the bed according to different degrees of freedom can be controlled by a motorized actuator, and/or a hydraulic or pneumatic actuator, or manually and can be executed at least partially simultaneously or in sequence and/or at partially overlapping times.

8. The system according to claim 1, further comprising a position fixing device of the patient's head to the bed that can be applied permanently or replaceable to the bed, said position fixing device being provided in combination with a receiving coil of MRI signals, said receiving coil of MRI signals being formed by a single element or by a plurality of elements housing conductors forming a coil circuit or conductor segments which are electrically connected or connectable to each other, the element or plurality of elements being removably attachable to at least part of structural elements of the position fixing device at predetermined points thereof so as to configure, in a condition attached to the position fixing device and in a connected state of said conductor segments, a receiving coil for the head.

9. The system according to claim 8, wherein some or all of the elements forming the receiving coil are stably hinged to the-a support base of the patient's head of said head fixing device or to other structural parts of said head fixing device, thereby housing said some or all of the elements and/or the support base of the patient's head and/or additional structural elements of the position fixing device equipped with receiver coil circuit segments that can oscillate in a position of mutual mechanical and electrical approach and connection in which the receiving coil is realized and in an open position, in which said some or all of the segments are mutually distant from each other and provide access to the patient's head.

10. The system according to claim 8, wherein the receiving coil has an annular shape and is provided with securing means to the head of the patient and/or to a craniostat, the securing means being selectable among clamping bands or belts, an inner diameter of the annular shape and a shape in plan thereof being configured to contain within an opening at least an operating field, making the operating field accessible even when the receiving coil is worn on the head of the patient.

11. The system according to claim 2, wherein said system is provided in combination with a surgical operation station provided at a predetermined distance from the imaging system, the surgical station comprising at least one unit for monitoring physiological parameters of the patient with sensors and/or probes applied to the patient and connected by cables to a control unit, and/or a unit for administering one or more drugs during surgery with injectors connected to the patient via tubes and needles,
wherein the bed being provided, along at least part of a surface extension thereof and/or along at least part of perimeter edges thereof, with removable fastenings or removable restraining elements of said cables and/or said tubes, said cables and/or said tubes being provided with a redundant length such as to enable said cables and/or said tubes to follow movements of the bed, including the rotation between said two extreme positions.

12. The system according to claim 2, wherein the extreme external position of the bed, at the end of which the patient's head positioning area is provided, is coinciding with an operating position in a surgical operation station, and wherein, at the extreme position of the patient's head positioning area and of the surgical operation station, an intensity of the magnetic field permeating out of a gantry of the magnetic structure is out of a 5 Gauss equipotential line of the magnetic field generated by the magnetic structure,
